SEQUENCE LISTING (2 sequences)

SEQ ID NO: 1 - protein, 707 aa, Homo sapiens
MNQELLSVGSKRRRTGGSLRGNPSSSQVDEEQMNRVVEEEQQQQLRQQEEEHTARNGEVVGVEPRPGGQNDSQQGQLEENNNRFISVDEDSSGNQEEQEEDEEHAGEQDEEDEEEEEMDQESDDFDQSDDSSREDEHTHTNSVTNSSSIVDLPVHQLSSPFYTKTTKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 2 - protein, 707 aa, synthetic construct
MNQELLSVGSKRRRTGGSLRGNPSSSQVDEEQMNRVVEEEQQQQLRQQEEEHTARNGEVVGVEPRPGGQNDSQQGQLEENNNRFISVDEDSSGNQEEQEEDEEHAGEQDEEDEEEEEMDQESDDFDQSDDSSREDEHTHTNSVTNSSSIVDLPVHQLSSPFYTKTTKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVCCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

(19) United States
(12) Patent Application Publication
(10) Pub. No.: US 2026/0137787 A1
(43) Pub. Date: May 21, 2026

(54) COMPOUNDS AND METHODS FOR FBXW7-R465C-MEDIATED PROTEIN DEGRADATION

(71) Applicant: Northwestern University, Evanston, IL (US)

(72) Inventors: Xiaoyu Zhang, Evanston, IL (US); Ananya Angela Basu, Evanston, IL (US)

(21) Appl. No.: 19/391,023

(22) Filed: Nov. 17, 2025

Related U.S. Application Data
(60) Provisional application No. 63/722,709, filed on Nov. 20, 2024.

(51) Int. Cl. A61K 47/55; A61P 35/00

(52) U.S. Cl. CPC A61K 47/55; A61P 35/00

(57) ABSTRACT

Provided herein compounds (PROTACs) targeting the mutant E3 ligase FBXW7-R465C and uses thereof.

### STATEMENT OF RELATED APPLICATIONS

This application claims priority to and the benefit of U.S. Provisional Patent Application No. 63/722,709, filed Nov. 20, 2024, the entire contents of which are incorporated herein by reference for all purposes.

### STATEMENT OF GOVERNMENT SUPPORT

This invention was made with government support under grant CA248715 awarded by The National Institutes of Health. The government has certain rights in the invention.

### SEQUENCE LISTING

The text of the computer readable sequence listing filed herewith, titled “NWEST-44044-202_SQL.xml”, created Nov. 17, 2025, having a file size of 4,202 bytes, is hereby incorporated by reference in its entirety.

### FIELD

The disclosure relates to PROTACs targeting the mutant E3 ligase FBXW7-R465C and uses thereof.

### BACKGROUND

Targeted protein degradation (TPD) is an emerging strategy that employs small molecules or biologics to direct proteins to proteolytic degradation machinery, such as the proteasome and lysosome, facilitating the removal of target proteins. In ubiquitin-proteasome system (UPS)-dependent TPD, heterobifunctional compounds, known as PROTACs (proteolysis-targeting chimeras), or monofunctional molecular glues, induce proximity between the target protein and an E3 ligase. This allows the E3 ligase to ubiquitinate the target protein, directing it for subsequent degradation by the proteasome. The E3 ligase family comprises over 600 members in humans, yet most TPD-susceptible proteins identified to date are degraded through only two: CRBN and VHL. As such, there remains a need for identifying TPD-compatible E3 ligases and the development of PROTACs targeting the same.

### SUMMARY

In some aspects, provided herein are proteolysis-targeting chimeras (PROTACs). In some embodiments, provided herein is a PROTAC comprising
a) a domain that binds to the mutant E3 ligase FBXW7-R465C;
b) a target-binding domain, and
c) a linker connecting the domain that binds to FBXW7-R465C to the target-binding domain.

In some embodiments, the domain that binds to FBXW7-R465C comprises the structure:

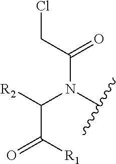

wherein R1 is selected from the following:

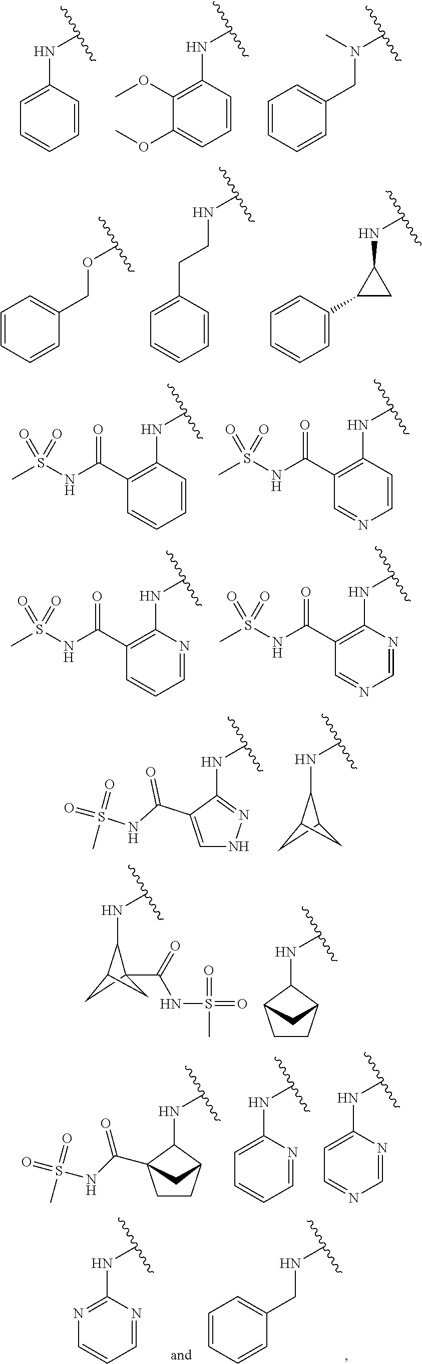

and wherein R2 is selected from the following;

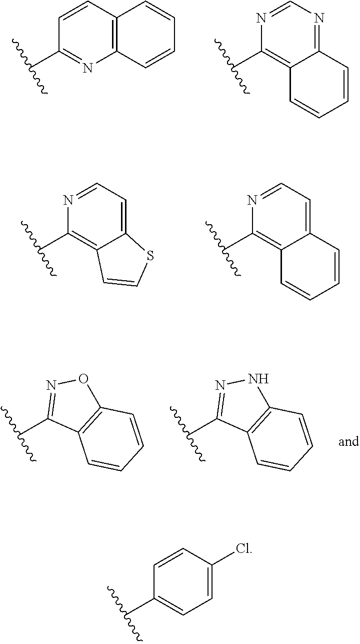

In some embodiments, the domain that binds to FBXW7-R465C comprises the structure:

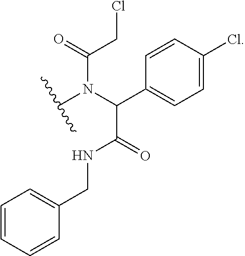 [SMILES: *N(C(=O)CCl)C(C(=O)NCc1ccccc1)c1ccc(Cl)cc1]

In some embodiments, the linker comprises one or more alkylene oxide units. In some embodiments, binding of the PROTAC to FBXW7-R465C and to the target induces FBXW7-R465C-mediated targeted protein degradation of the target.

In some embodiments, the target is 12-kDa FK506-binding protein (FKBP12). For example, in some embodiments, the target-binding domain comprises the structure:

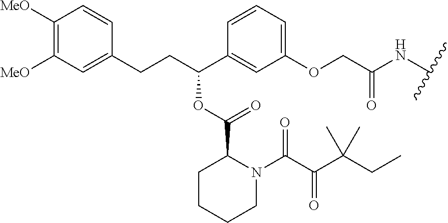 [SMILES: *NC(=O)COc1cccc([C@@H](CCc2ccc(OC)c(OC)c2)OC(=O)[C@@H]2CCCCN2C(=O)C(=O)C(C)(C)CC)c1]

In some embodiments, the PROTAC is 10-SLF, defined by the structure:

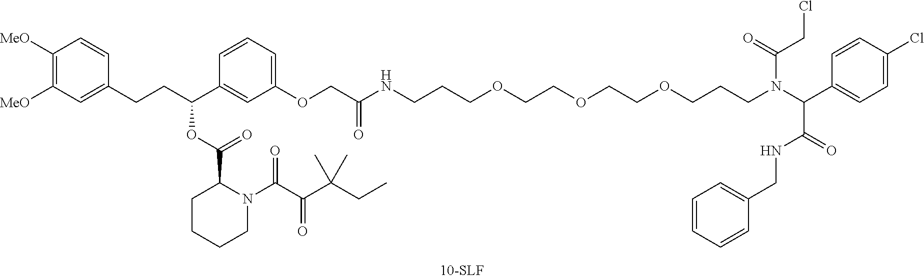 [SMILES: CCC(C)(C)C(=O)C(=O)N1CCCC[C@H]1C(=O)O[C@H](CCc1ccc(OC)c(OC)c1)c1cccc(OCC(=O)NCCCOCCOCCOCCCN(C(=O)CCl)C(C(=O)NCc2ccccc2)c2ccc(Cl)cc2)c1]

In some embodiments, the target-binding domain binds to one or more kinases. For example, in some embodiments the target-binding domain comprises the structure:

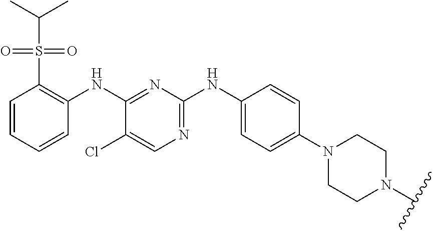 [SMILES: *N1CCN(c2ccc(Nc3ncc(Cl)c(Nc4ccccc4S(=O)(=O)C(C)C)n3)cc2)CC1]

In some embodiments, the PROTAC is 10-MKI, defined by the structure:

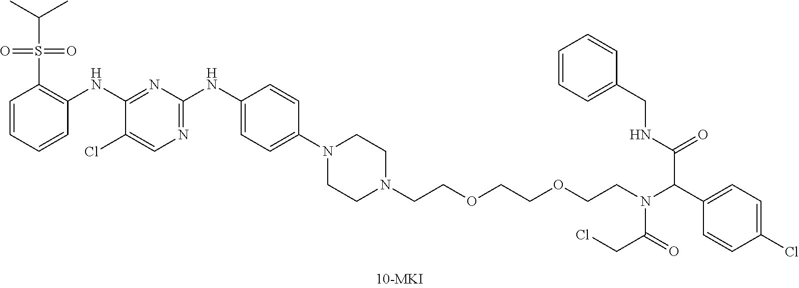 [SMILES: CC(C)S(=O)(=O)c1ccccc1Nc1nc(Nc2ccc(N3CCN(CCOCCOCCN(C(=O)CCl)C(C(=O)NCc4ccccc4)c4ccc(Cl)cc4)CC3)cc2)ncc1Cl]

In some embodiments, the target is bromodomain-containing protein 4 (BRD4). For example, in some embodiments the target-binding domain comprises the structure:

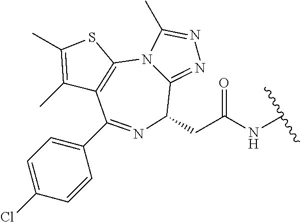 [SMILES: *NC(=O)C[C@@H]1N=C(c2ccc(Cl)cc2)c2c(sc(C)c2C)-n2c(C)nnc21]

In some embodiments, the target is anaplastic lymphoma kinase (ALK). For example, in some embodiments the target-binding domain comprises the structure:

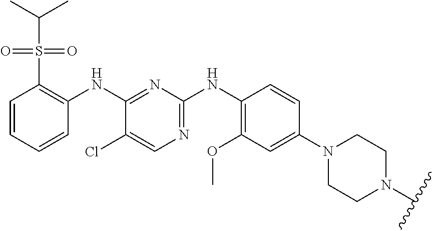 [SMILES: *N1CCN(c2ccc(Nc3ncc(Cl)c(Nc4ccccc4S(=O)(=O)C(C)C)n3)c(OC)c2)CC1]

In some aspects, provided herein are methods of targeted protein degradation in a cell or a subject comprising administering to the cell or subject a PROTAC described herein. In some embodiments, the subject has a neurological disease or cancer.

### BRIEF DESCRIPTION OF THE DRAWINGS

FIGS. 1A-1C show identification of heterobifunctional compounds that degrade FKBP12 in an FBXW7-R465C-dependent manner. FIG. 1A shows analysis of acquired cysteines within 680 human E3 ligases. FIG. 1B shows the TCGA hotspot count of the 39 acquired cysteines occurring in 35 E3 ligases. FIG. 1C shows screening of an FKBP12-directed heterobifunctional compound library to identify compounds that degrade FKBP12 in an FBXW7-R465C-dependent manner.

FIGS. 2A-2D show FBXW7-R465C supports 10-SLF-induced ubiquitination and degradation of FKBP12. FIG. 2A shows structures of 10-SLF and P10-SLF. FIG. 2B shows dose-dependent degradation of FKBP12_NLS by 10-SLF. HEK293T cells expressing HA-FBXW7 WT or R465C and FLAG-FKBP12_NLS were treated with 0.1-1 μM 10-SLF for 8 hours. The bar graph represents quantification of the FKBP12/β-Actin protein content. Data are presented as the mean values (n=2 independent replicates). FIG. 2C shows 10-SLF-induced FKBP12_NLS degradation was blocked by MG132, SLF, and MLN4924. The bar graph represents quantification of the FKBP12/β-Actin protein content. Data are presented as the mean values (n=2 independent replicates). FIG. 2D shows time-course study of 10-SLF-induced FKBP12_NLS degradation. The bar graph represents quantification of the FKBP12/β-Actin protein content. Data are presented as the mean values (n=2 independent replicates).

FIGS. 3A-3F show mechanistic insights into FBXW7-R465C-mediated target degradation. FIG. 3A shows genomic PCR confirming FBXW7^{R465C} KO in AsPC-1 cells. FIG. 3B shows 10-SLF induced FKBP12_NLS degradation in AsPC-1 WT but not FBXW7^{R465C} KO cells. The bar graph represents quantification of the FKBP12/HSP90 protein content. Data are presented as the mean values ±s.e.m. (n=3 independent replicates). FIG. 3C shows P10-SLF did not induce FKBP12_NLS degradation in either AsPC-1 WT or FBXW7^{R465}C KO cells. The bar graph represents quantification of the FKBP12/HSP90 protein content. Data are presented as the mean values ±s.e.m. (n=3 independent replicates). FIG. 3D shows a series of mutations were introduced in FBXW7 for this study. The crystal structure was obtained from the Protein Data Bank (PDB: 7T1Y). The bottom panel shows the evaluation of different FBXW7 mutations in supporting 10-SLF-induced FKBP12 degradation. The bar graph represents quantification of the FKBP12/β-Actin protein content. Data are presented as the mean values (n=2 independent replicates). FIG. 3E shows the modeling study revealed interactions between the electrophilic portion of 10-SLF and a pocket in FBXW7 involving R465C. FIG. 3F shows FBXW7-R505C did not support the degradation of FKBP12_NLS induced by 10-SLF. The bar graph represents quantification of the FKBP12/β-Actin protein content. Data are presented as the mean values (n=2 independent replicates).

FIGS. 4A-4B show 10-SLF induces the formation of a ternary complex between FKBP12 and FBXW7-R465C. FIG. 4A shows coimmunoprecipitation assays demonstrated that HA-FBXW7-R465C and HA-FBXW7-R465C/C466A coimmunoprecipitated with FLAG-FKBP12_NLS in the presence of 10-SLF and MG132. The bar graph represents quantification of the immunoprecipitated HA-FBXW7 compared to HA-FBXW7 in whole cell lysates (WCL). Data are presented as the mean values (n=2 independent replicates). FIG. 4B shows FKBP12 enrichment proteomic analysis revealed that HA-FBXW7-R465C and HA-FBXW7-R465C/C466A interacted with FLAG-FKBP12_NLS in cells treated with 10-SLF.

FIGS. 5A-5D show use of FBXW7-R465C for the degradation of additional protein targets. FIG. 5A shows the structure of 10-MKI. FIG. 5B shows global proteomic analysis in AsPC-1 parental and FBXW7^{R465}C KO cells treated with 5 M 10-MKI for 24 hours. Data are presented as the mean values (n=2 independent replicates for DMSO treatment, n=3 independent replicates for 10-MKI treatment). FIG. 5C shows treatment with 10-MKI (5 M, 24 hours) induced AURKA degradation in AsPC-1 parental cells but not in FBXW7^{R465C} KO cells. The bar graph represents quantification of the AURKA/HSP90 protein content. Data are presented as the mean values ±s.e.m. (n=3 independent replicates). FIG. 5D shows 10-MKI induced the degradation of AURKA and CSNK1D in CCRF-CEM cells, but not in HEK293T or A549 cells. The bar graph represents quantification of the AURKA/HSP90 protein content. Data are presented as the mean values ±s.e.m. (n=3 independent replicates).

FIGS. 6A-6B show identification of heterobifunctional compounds that degrade FKBP12_NLS in an FBXW7-R465C-dependent manner. FIG. 6A shows genomic PCR confirming FBXW7 KO in HEK293T cells. FIG. 6B shows western blot analysis of a focused FKBP12-directed heterobifunctional compound library to identify compounds that induce FKBP12_NLS degradation in an FBXW7-R465C-dependent manner.

FIG. 7 shows the crystal structure indicating that R505 is positioned near R465.

### DETAILED DESCRIPTION

### 1. Definitions

Although any methods and materials similar or equivalent to those described herein can be used in the practice or testing of embodiments described herein, some preferred methods, compositions, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to ±10% of the indicated number. For example, “about 10%” may indicate a range of 9% to 11%, and “about 1” may mean from 0.9-1.1. Other meanings of “about” may be apparent from the context, such as rounding off; for example, “about 1” may also mean from 0.5 to 1.4.

As used herein, the terms “comprise”, “include”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof).

An “effective amount” refers to an amount sufficient to elicit a desired biological response (e.g., treating a condition). As will be appreciated by those skilled in the art, the effective amount may vary depending on such factors as the desired biological endpoint, the pharmacokinetics, the condition being treated, the mode of administration, and the age and health of the subject. An effective amount encompasses therapeutic and prophylactic treatment. For example, a “therapeutically effective amount” is an amount sufficient to provide a therapeutic benefit in the treatment of a condition, or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

### 2. Proteolysis Targeting Chimeras

In some aspects, provided herein are compounds. In some embodiments, the compounds are proteolysis-targeting chimeras, or PROTACs. As used herein, the term “proteolysis-targeting chimera” or “PROTAC” are used interchangeably and refer to a heterobifunctional molecule comprising three components: a target-binding domain (also referred to as a “warhead”), a linker, and an E3 ubiquitin ligase binding domain (also referred to as an “anchor”). The linker connects the target-binding domain to the E3 ubiquitin ligase binding domain. PROTACs achieve degradation of a target protein when the target-binding domain binds to the target protein and the E3 ubiquitin ligase binding domain binds to an E3 ubiquitin ligase, resulting in ubiquitylation of the target protein and subsequent degradation thereof by the proteasome. This process is referred to as “targeted protein degradation” or “TPD”.

E3 ubiquitin ligases, also referred to herein as E3 ligases or ubiquitin ligases, refer to a family of proteins that enable movement of ubiquitin from a ubiquitin carrier (e.g. an E2 ubiquitin-conjugating enzyme) to a substrate (e.g. a protein substrate). E3 ligases are involved in many processes including protein degradation, DNA repair, cell cycle progression, transcriptional regulation, signal transduction, and the like. In some embodiments, the PROTAC comprises a domain that binds to the mutant E3 ligase FBXW7-R465C.

FBXW7 is an F-box protein that acts as a substrate recognition unit of the SCF E3 ubiquitin-protein ligase complex. FBXW7 is a typical tumor suppressor that targets a large number of critical oncoprotein substrates including cyclic E, c-JUN, cMYC, NOTCH-1, MLC-1, and the like for ubiquitylation and proteasome degradation. Mutations in FBXW7 can disrupt this function, and are thus associated with various cancer types.

In some embodiments, FBXW7 refers to a protein comprising the following amino acid sequence:

(SEQ ID NO: 1)
MNQELLSVGSKRRRTGGSLRGNPSSSQVDEEQMNRVVEEEQQQQLRQQE
EEHTARNGEVVGVEPRPGGQNDSQQGQLEENNNRFISVDEDSSGNQEEQ
EEDEEHAGEQDEEDEEEEEMDQESDDFDQSDDSSREDEHTHTNSVTNSS
SIVDLPVHQLSSPFYTKTTKMKRKLDHGSEVRSFSLGKKPCKVSEYTST
TGLVPCSATPTTFGDLRAANGQGQQRRRITSVQPPTGLQEWLKMFQSWS
GPEKLLALDELIDSCEPTQVKHMMQVIEPQFQRDFISLLPKELALYVLS
FLEPKDLLQAAQTCRYWRILAEDNLLWREKCKEEGIDEPLHIKRRKVIK
PGFIHSPWKSAYIRQHRIDTNWRRGELKSPKVLKGHDDHVITCLQFCGN
RIVSGSDDNTLKVWSAVTGKCLRTLVGHTGGVWSSQMRDNIIISGSTDR
TLKVWNAETGECIHTLYGHTSTVRCMHLHEKRVVSGSRDATLRVWDIET
GQCLHVLMGHVAAVRCVQYDGRRVVSGAYDFMVKVWDPETETCLHTLQG
HTNRVYSLQFDGIHVVSGSLDTSIRVWDVETGNCIHTLTGHQSLTSGME
LKDNILVSGNADSTVKIWDIKTGQCLQTLQGPNKHQSAVTCLQFNKNFV
ITSSDDGTVKLWDLKTGEFIRNLVTLESGGSGGVVWRIRASNTKLVCAV
GSRNGTEETKLLVLDFDVDMK.

FBXW7-R465C is a FBXW7 mutant containing a substitution mutation (arginine to cysteine) at position 465 relative to SEQ ID NO: 1. Position 465 in SEQ ID NO: 1 is highlighted and underlined. Accordingly, FBXW7-R465C refers to a protein comprising the following amino acid sequence:

(SEQ ID NO: 2)
MNQELLSVGSKRRRTGGSLRGNPSSSQVDEEQMNRVVEEEQQQQLRQQE
EEHTARNGEVVGVEPRPGGQNDSQQGQLEENNNRFISVDEDSSGNQEEQ
EEDEEHAGEQDEEDEEEEEMDQESDDFDQSDDSSREDEHTHTNSVTNSS
SIVDLPVHQLSSPFYTKTTKMKRKLDHGSEVRSFSLGKKPCKVSEYTST
TGLVPCSATPTTFGDLRAANGQGQQRRRITSVQPPTGLQEWLKMFQSWS
GPEKLLALDELIDSCEPTQVKHMMQVIEPQFQRDFISLLPKELALYVLS
FLEPKDLLQAAQTCRYWRILAEDNLLWREKCKEEGIDEPLHIKRRKVIK
PGFIHSPWKSAYIRQHRIDTNWRRGELKSPKVLKGHDDHVITCLQFCGN
RIVSGSDDNTLKVWSAVTGKCLRTLVGHTGGVWSSQMRDNIIISGSTDR
TLKVWNAETGECIHTLYGHTSTVCCMHLHEKRVVSGSRDATLRVWDIET
GQCLHVLMGHVAAVRCVQYDGRRVVSGAYDFMVKVWDPETETCLHTLQG
HTNRVYSLQFDGIHVVSGSLDTSIRVWDVETGNCIHTLTGHQSLTSGME
LKDNILVSGNADSTVKIWDIKTGQCLQTLQGPNKHQSAVTCLQFNKNFV
ITSSDDGTVKLWDLKTGEFIRNLVTLESGGSGGVVWRIRASNTKLVCAV
GSRNGTEETKLLVLDFDVDMK.

In some embodiments, provided herein is a proteolysis-targeting chimera (PROTAC) comprising:
a) domain that binds to the mutant E3 ligase FBXW7-R465C;
b) a target-binding domain, and
c) a linker connecting the domain that binds to FBXW7-R465C to the target-binding domain.

In some embodiments, the domain that binds to FBXW7-R465C comprises the structure:

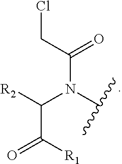

In some embodiments, R1 is selected from the following:

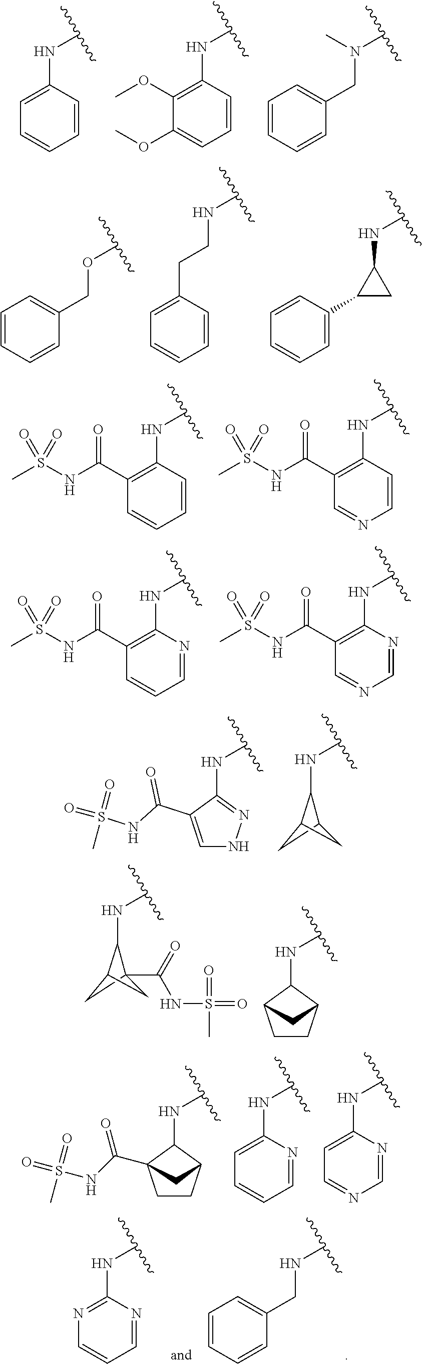

In some embodiments, R2 is selected from the following:

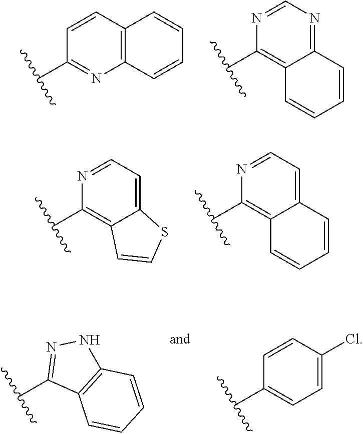

In some embodiments, the domain that binds to FBXW7-R465C comprises the structure:

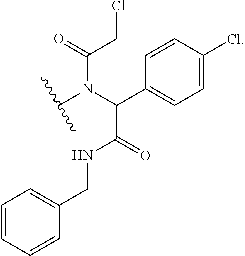 [SMILES: *N(C(=O)CCl)C(C(=O)NCc1ccccc1)c1ccc(Cl)cc1]

As used herein, in chemical structures the indication:

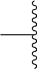

represents a point of attachment of one moiety to another moiety. For example, the indication may represent a point of attachment of the domain that binds to FBXW7-R465C to the linker (e.g. the linker that connects the domain that binds to FBXW7-R465C to the target-binding domain). As another example, the indication may represent a point of attachment of the target-binding domain to the linker (e.g. the linker that connects the target-binding domain to the domain that binds to FBXW7-R465C).

Any suitable linker may be used. In some embodiments, the linker comprises a combination of one or more chemical motifs (e.g. units or atoms). Exemplary linker motifs (units) include, for example, PEG, alkyl, glycol, alkyne, triazole, piperazine, and piperidine motifs. In some embodiments, the linker comprises a single type of chemical motif (e.g. unit). In some embodiments, the linker comprises multiple different types of chemical motifs (e.g. units). In some embodiments, the linker comprises one or more alkylene oxide units. For example, in some embodiments, the linker comprises one or more ethylene oxide units. Generally speaking, the linker should be of a suitable length to achieve a sufficient distance between the target-binding domain (e.g. the warhead) and the domain that binds to FBXW7-R465C to avoid steric repulsions when the target protein and to FBXW7-R465C are both bound to the PROTAC, while not being too long such that the PROTAC fails to facilitate ubiquitylation and degradation of the target. In some embodiments, the linker comprises 3 to 50 units. In some embodiments, the linker comprises 3 to 40 units, 3 to 30 units, 3 to 25 units, 3 to 20 units, 3 to 15 units, or 3 to 10 units. In some embodiments, the linker is a flexible linker.

In some embodiments, the linker comprises one or more alkylene glycol repeat units, such as ethylene glycol or propylene glycol repeat units. For example, in some embodiments, the linker comprises a poly- or oligo-ethylene glycol chain:

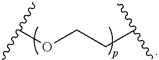

In some embodiments, p is 1 to 30. In some embodiments, p is 1 to 20. In some embodiments, p is 1 to 10. In some embodiments, p is 1 to 6. For example, in some embodiments p is 1, 2, 3, 4, 5, or 6.

In some embodiments, the linker comprises a group of formula:

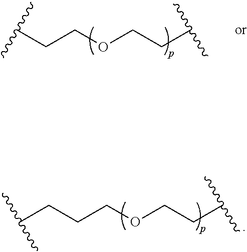

In some embodiments, p is 1 to 30. In some embodiments, p is 1 to 20. In some embodiments, p is 1 to 10. In some embodiments, p is 1 to 6. For example, in some embodiments, p is 1, 2, 3, 4, 5, or 6.

The target-binding domain may bind to any suitable target protein for which targeted protein degradation (TPD) is desired. In some embodiments, the target protein is 12-kDa FK506-binding protein (FKBP12). FKBP12 is a member of the FK506 binding protein (FKBP) family. In some embodiments, the target protein is human FKBP12.

In some embodiments, the target protein is FKBP12 and the target-binding domain comprises the structure:

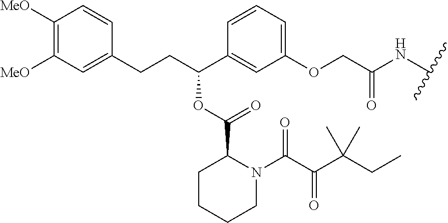 [SMILES: *NC(=O)COc1cccc([C@@H](CCc2ccc(OC)c(OC)c2)OC(=O)[C@@H]2CCCCN2C(=O)C(=O)C(C)(C)CC)c1]

In some embodiments, the PROTAC is 10-SLF, defined by the structure:

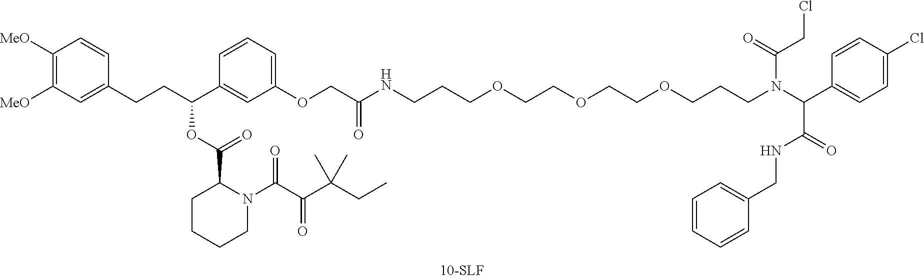 [SMILES: CCC(C)(C)C(=O)C(=O)N1CCCC[C@H]1C(=O)O[C@H](CCc1ccc(OC)c(OC)c1)c1cccc(OCC(=O)NCCCOCCOCCOCCCN(C(=O)CCl)C(C(=O)NCc2ccccc2)c2ccc(Cl)cc2)c1]

In some embodiments, the target-binding domain binds to one or more kinases. A PROTAC as described herein comprising a target-binding domain that binds to multiple kinases is also referred to herein as a multiple kinase inhibitor, or MKI. MKIs may target a wide variety of kinases including intracellular kinases and/or cell surface kinases. Exemplary kinases that may targeted by multiple kinase inhibitors include protein kinases (e.g. serine/threonine kinases, tyrosine kinases, histidine kinases, dual-specificity protein kinases), lipid kinases, and carbohydrate kinases. In some embodiments, the MKI targets one or more kinases that are dysregulated in cancer. For example, in some embodiments the MKI targets one or more of EGFR, ALK, Src family kinases, Jak family kinases, cyclin-dependent kinases (CDKs), KIT kinases, aurora kinases (e.g. AURKA, AURKB, AURKC), casein kinases (e.g. CSNK1D), Akt, MAPKs, cRAF, IKB kinases, BRAF, etc.

In some embodiments, the target-binding comprises the structure:

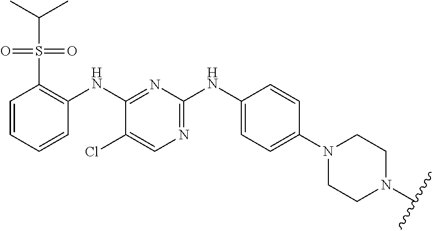 [SMILES: *N1CCN(c2ccc(Nc3ncc(Cl)c(Nc4ccccc4S(=O)(=O)C(C)C)n3)cc2)CC1]

In some embodiments, the PROTAC is 10-MKI, defined by the structure:

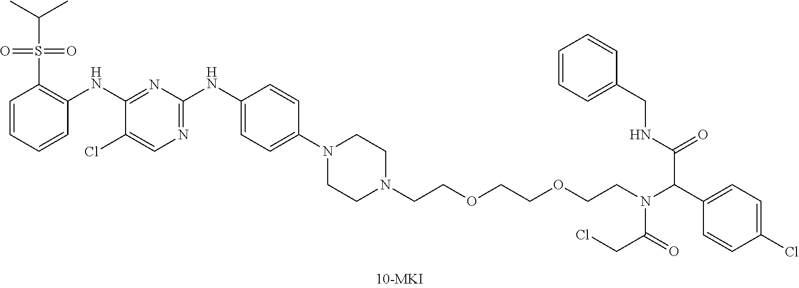 [SMILES: CC(C)S(=O)(=O)c1ccccc1Nc1nc(Nc2ccc(N3CCN(CCOCCOCCN(C(=O)CCl)C(C(=O)NCc4ccccc4)c4ccc(Cl)cc4)CC3)cc2)ncc1Cl]

In some embodiments, the target protein is Bromodomain-containing protein 4 (BRD4). BRD4 is a member of the bromodomain and extraterminal domain (BET) family which also includes BRD2, BRD3, and BRDT. BRD4 contains two bromodomains that recognize acetylated lysine residues. BRD4 is a transcriptional and epigenetic regulator and has been implicated in embryogenesis, cell regulation, and cancer development.

In some embodiments, the target protein is BRD4 and the target-binding domain comprises the following structure:

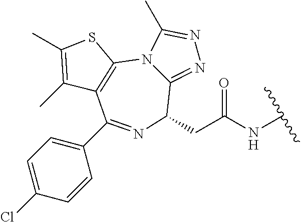 [SMILES: *NC(=O)C[C@@H]1N=C(c2ccc(Cl)cc2)c2c(sc(C)c2C)-n2c(C)nnc21]

In some embodiments, the target protein is anaplastic lymphoma kinase (ALK). ALK is a receptor tyrosine kinase involved in regulation of cellular growth and differentiation. ALK is implicated in multiple cancer types, including lymphoma and non-small cell lung cancer.

In some embodiments, the target protein is ALK and the target-binding domain comprises the following structure:

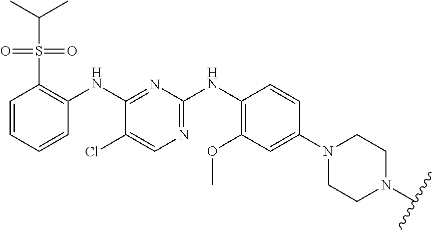 [SMILES: *N1CCN(c2ccc(Nc3ncc(Cl)c(Nc4ccccc4S(=O)(=O)C(C)C)n3)c(OC)c2)CC1]

As evidenced by the multiple exemplary PROTACs described above and in the accompanying Examples, provided herein is a PROTAC platform that can be designed to target any desired target protein to facilitate targeted protein degradation thereof. The target-binding domain is not limited to the exemplary target-binding domain structures provided above, rather the domain that binds to FBXW7-R465C can be attached (via the linker) to any suitable target-binding domain. For example, the target protein may be implicated in a disease or condition. For example, the target protein may be overexpressed in a disease or condition, and targeted protein degradation of the target protein may be desired to reduce the levels of the target and thereby treat the disease or condition. In some embodiments, the target protein is implicated in a neurological disease or condition, such as Alzheimer's disease or Parkinson's disease. In some embodiments, the target protein is implicated in cancer. In some embodiments, the target protein lacks functional sites for other forms of therapeutics (e.g. small molecules, antibodies, etc.).

### 3. Methods of Use

In some embodiments, the PROTACs herein provided herein find use in methods for targeted protein degradation in a cell or in a subject. In some embodiments, provided herein is a method for targeted protein degradation in a cell or a subject, the method comprising providing to the cell or subject a PROTAC described herein. The PROTAC herein induces or promotes targeted protein degradation of the target protein, thereby reducing levels of the target protein in the cell or subject. As such, a “method for targeted protein degradation” refers to a method involving administration of a PROTAC herein to induce or promote targeted protein degradation of a desired target protein in a cell or a subject (e.g. a protein that is targeted by the target-binding domain of the PROTAC).

The target protein may be implicated in a disease or condition, and as such TPD of the target protein (e.g. which results in decreased levels of the target protein) may treat the disease or condition. As such, in some embodiments provided herein are methods for TPD in a subject afflicted with a particular disease or condition for which TPD of the target protein is beneficial or therapeutic. In some embodiments, the disease or condition is a neurological disease or condition, such as Alzheimer's disease or Parkinson's disease. In some embodiments, the disease is cancer. In some embodiments, the disease is cancer and the subject is identified as having a R465C mutation in FBXW7.

In some embodiments, the target protein is FKBP12, one or more kinases, BRD4, or ALK. In some embodiments, the PROTAC is 10-SLF or 10-MKI. As described above, these exemplary PROTACs are not to be construed as limiting and any suitable PROTAC comprising the domain that binds to FBXW7-R465C attached to a target-binding domain via a linker can be designed and implemented in methods for targeted degradation of a desired target protein.

In some embodiments, provided herein is a method for targeted protein degradation of FKBP12 in a cell or a subject. In some embodiments, comprising providing to the cell or subject a PROTAC comprising the domain that binds FBXW7-R465C conjugated to a target binding domain that binds to FKBP12 (e.g. the PROTAC 10-SLF). FKBPs are implicated in some neurodegenerative diseases, such as Alzheimer's disease (AD) and Parkinson's disease (PD). In some embodiments, the subject has a neurodegenerative disease, such as AD or PD. In some embodiments, provided herein is a method for TPD of FKBP12 in a subject having or suspected of having a neurodegenerative disease, comprising providing to the subject a PROTAC provided herein targeting FKBP12 (e.g. 10-SLF). In some embodiments provided herein are methods of treating a neurodegenerative disease in subject, comprising providing to the subject a composition comprising 10-SLF.

In some embodiments, provided herein is a method for targeted protein degradation of one or more kinases in a cell or a subject. In some embodiments, comprising providing to the cell or subject a PROTAC comprising the domain that binds FBXW7-R465C conjugated to a target binding domain that binds to one or more kinases (e.g. the PROTAC 10-MKI). In some embodiments, the PROTAC induces degradation of one or more protein kinases (e.g. serine/threonine kinases, tyrosine kinases, histidine kinases, dual-specificity protein kinases), lipid kinases, and/or carbohydrate kinases. In some embodiments, the PROTAC induces degradation of one or more kinases dysregulated in cancer, for example EGFR, ALK, Src family kinases, Jak family kinases, cyclin-dependent kinases (CDKs), KIT kinases, aurora kinases (e.g. AURKA, AURKB, AURKC), casein kinases (e.g. CSNK1D), Akt, MAPKs, cRAF, IKB kinases, BRAF, etc.

In some embodiments, provided herein is a method for targeted protein degradation of BRD4 in a cell or a subject, comprising providing to the cell or subject a PROTAC comprising the domain that binds to FBXW7-R465C attached to a domain that binds BRD4. BRD4 is a regulator of cancer cell proliferation. In some embodiments, the subject has cancer. In some embodiments, provided herein is a method for TPD of BRD4 in a subject having or suspected of having cancer, comprising providing to the subject a PROTAC provided herein targeting BRD4.

In some embodiments, provided herein is a method for targeted protein degradation of ALK in a cell or a subject. ALK is implicated in various cancers. In some embodiments, the subject has cancer. In some embodiments, provided herein is a method for TPD in a subject having or suspected of having cancer, comprising providing to the subject a PROTAC provided herein targeting ALK.

The PROTAC may be provided to the subject by any suitable administration route. Suitable administration routes include, for example, oral administration and parenteral administration (e.g. by injection, such as intravenous, intraarterial, intradermal, subcutaneous, intramuscular, etc.).

In some embodiments, the PROTAC is comprised in a composition (e.g. a pharmaceutical composition) comprising a pharmaceutically acceptable carrier or excipient. Reference to providing or administering the PROTAC to the subject is inclusive of providing or administering a composition (e.g. a pharmaceutical composition) comprising the PROTAC to the subject. The phrase “pharmaceutically acceptable,” as used in connection with compositions of the present disclosure, refers to molecular entities and other ingredients of such compositions that are physiologically tolerable and do not typically produce undesirable reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. The pharmaceutically acceptable carrier should also be compatible with the active ingredient of the composition (e.g., the PROTAC). Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

The dose (e.g. the effective amount, the therapeutically effective amount) of the PROTAC provided to the subject may depend on the severity of the condition, the individual patient parameters including age, physical condition, size, gender and weight, the duration of the treatment, the nature of concurrent therapy (if any), the specific route of administration, the precise PROTAC used, and like factors within the knowledge and expertise of the health practitioner. In some embodiments, the effective amount is sufficient to treat a disease or condition (e.g. Alzheimer's Disease, Parkinson's Disease, cancer) in the subject. For example, in some embodiments the effective amount alleviates, relieves, ameliorates, improves, reduces the symptoms, or delays the progression of the disease or condition in the subject. For example, in some embodiments the effective amount induces a TPD of a protein implicated in the disease or condition, thereby treating the disease or condition in the subject.

It will be appreciated that appropriate dosages can vary from patient to patient. Determining the optimal dosage will generally involve the balancing of the level of therapeutic benefit against any risk or deleterious side effects of the treatments of the present disclosure. The amount and route of administration will ultimately be at the discretion of the physician, although generally the dosage will be to achieve local concentrations at the site of action which achieve the desired effect without causing substantial harmful or deleterious side-effects.

The PROTAC may be provided to the subject in be in a single dose or in multiple doses throughout the course of treatment. Methods of determining the most effective means and dosage of administration will vary with the exact PROTAC and route of administration used for therapy, the severity of the disease or condition, and the subject being treated. Single or multiple administrations can be carried out with the dose level and pattern being selected by the treating physician.

In some embodiments, a given dose is provided to the subject continuously or intermittently over the course of a suitable dosing window. For example, the dosing window may be 10 minutes to 6 hours, 20 minutes to 5 hours, 30 minutes to 4 hours, or about 1 to 3 hours. In some embodiments, the PROTAC is provided to the subject once per day. In some embodiments, the PROTAC is provided to the subject multiple times per day. In some embodiments, the PROTAC is provided to the subject every other day, every 3 days, every 4 days, every 5 days, every 6 days, every 7 days, every 8 days, every 9 days, every 10 days, every 2 weeks, every 3 weeks, monthly, every 2 months, every 3 months, every 4 months, every 5 months, every 6 months, annually, etc. The PROTAC may be administered until a desired reduction of symptoms is achieved.

In some embodiments, the PROTAC is provided to the subject in combination with other therapies for the disease or condition. Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

### EXAMPLES

### Example 1

Targeted protein degradation (TPD) is a pharmacological strategy that eliminates specific proteins from cells by harnessing cellular proteolytic degradation machinery. In proteasome-dependent TPD, expanding the repertoire of E3 ligases compatible with this approach could enhance the applicability of this strategy across various biological contexts. In this study, it was discovered that the somatic mutant of FBXW7, R465C, can be exploited by heterobifunctional compounds for targeted protein degradation. This work demonstrates the potential of utilizing mutant E3 ligases that occur exclusively in diseased cells for TPD applications.

### Results

Identification of Heterobifunctional Compounds that Degrade FKBP12 in an FBXW7-R465C-Dependent Manner

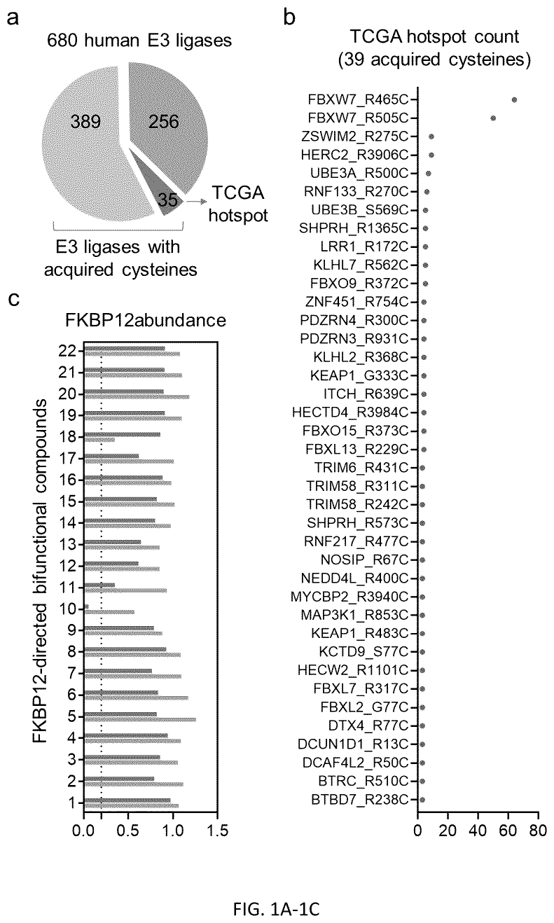

To investigate acquired cysteines in E3 ligases-some of which may exist in a reactive state amenable to ligand discovery—a set of 680 human E3 ligases was examined, 24,834 acquired cysteines occurring in 11,353 proteins cataloged in The Cancer Cell Line Encyclopedia (CCLE) were analyzed (Ghandi, M. et al. Next-generation characterization of the Cancer Cell Line Encyclopedia. Nature 569, 503-508 (2019). This analysis revealed 1,102 acquired cysteines present on 424 E3 ligases (FIG. 1a). By further filtering for hotspot mutations from The Cancer Genome Atlas (TCGA), 39 acquired cysteines in 35 E3 ligases were identified (FIG. 1a). Ranking these acquired cysteines by mutation frequency showed that the top two, R465C and R505C, are found in the same E3 ligase, FBXW7 (FIG. 1b).

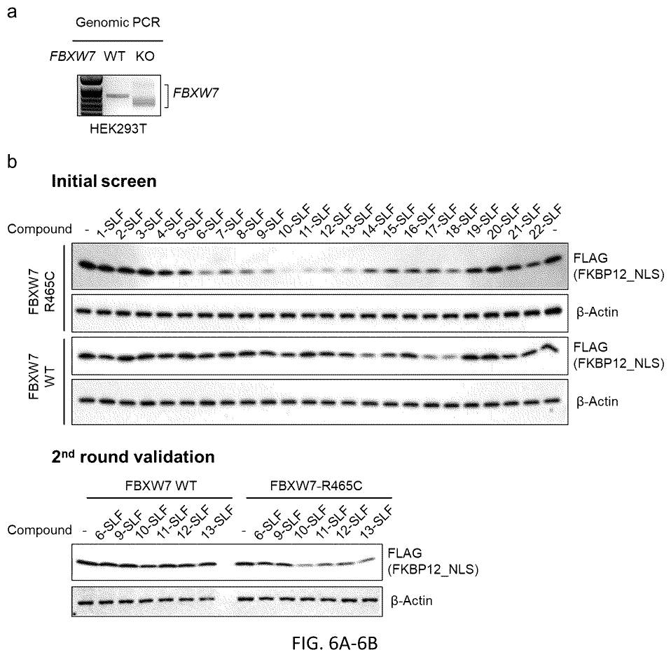

FBXW7, a member of the F-box protein family, functions as a component of the Cullin-RING ubiquitin ligase 1 (CRL1) complex. FBXW7 plays a role in regulating cell survival, proliferation, tumor invasion, DNA repair, and genomic stability, establishing its importance in oncogenesis. The R465 and R505 hotspot mutations are located in the WD40 domain and were initially believed to cause loss-of-function effects. Given that R465C is a more prevalent acquired cysteine according to CCLE hotspot mutations, this mutant was the focus for degrader discovery. FBXW7 was knocked out in HEK293T cells (FIG. 6a), then FLAG-tagged nucleus-localized FKBP12 (FLAG-FKBP12_NLS) was stably expressed along with either wild-type (WT) or R465C mutant HA-FBXW7. As the major FBXW7 isoform is nuclear, nucleus-localized FKBP12 was used for the screen. Cells were treated with 22 different FKBP12-targeting bifunctional compounds and FKBP12 abundance was measured in cells expressing either WT or R465C FBXW7 by Western blot. The results indicate that one compound, 10-SLF, selectively reduced FKBP12 levels in cells expressing FBXW7-R465C, but not in those expressing WT (FIG. 1c and FIG. 6b).

### FBXW7-R465C Supports 10-SLF-Induced Ubiquitination and Degradation of FKBP12

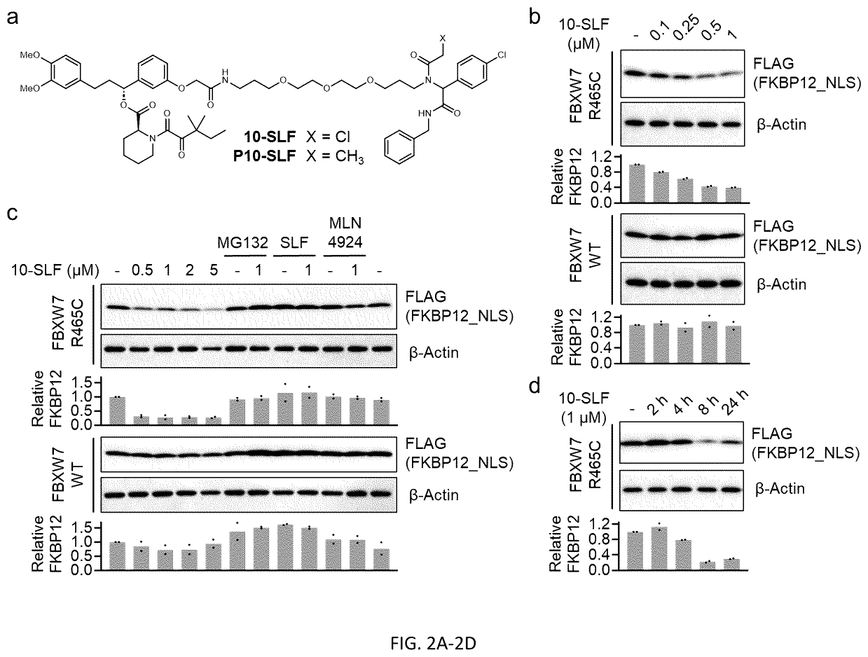

10-SLF is a bifunctional compound containing an a-chloroacetamide moiety (FIG. 2a) that may covalently interact with FBXW7-R465C. Different concentrations of 10-SLF were tested in HEK293T cells overexpressing FLAG-FKBP12_NLS and either HA-FBXW7 WT or R465C, observing a reduction in FKBP12_NLS levels starting at 0.25 μM only in cells expressing FBXW7-R465C, but not in those expressing WT FBXW7 (FIG. 2b). The 10-SLF-induced FKBP12_NLS reduction was blocked by MG132 (a proteasome inhibitor), SLF (a FKBP12 ligand), and MLN4924 (a neddylation inhibitor) (FIG. 2c), suggesting that FKBP12 degradation involves both the proteasome and Cullin-RING ligase pathways, consistent with FBXW7's role in the Cullin-RING E3 ligase family. A time-course study of 10-SLF-induced FKBP12_NLS degradation revealed maximal degradation after 8 hours (FIG. 2d).

Mechanistic Insights into FBXW7-R465C-Mediated Target Degradation

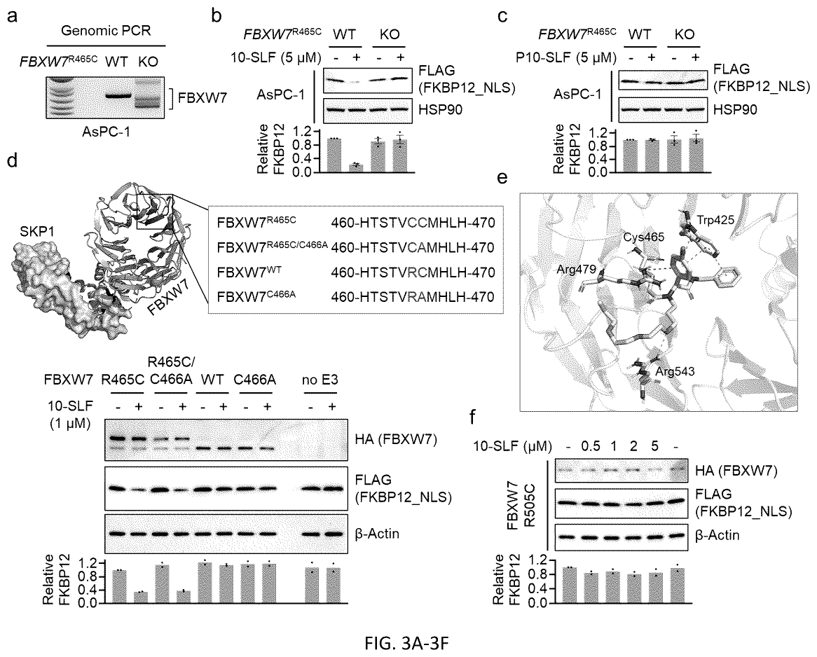

Next, it was investigated whether endogenously expressed FBXW7-R465C supports 10-SLF-induced FKBP12 degradation. AsPC-1, a human pancreatic tumor cell line that expresses FBXW7^{R465}C, was selected. FBXW7^{R465C} was knocked out in AsPC-1 cells (FIG. 3a), and subsequently FLAG-FKBP12_NLS was stably overexpressed to assess target degradation by 10-SLF. 10-SLF induced FKBP12 degradation in parental AsPC-1 cells but not in FBXW7^{R465C} knockout (KO) cells (FIG. 3b). The non-covalent variant, P10-SLF, did not induce FKBP12 degradation in either parental or KO cells (FIG. 3c).

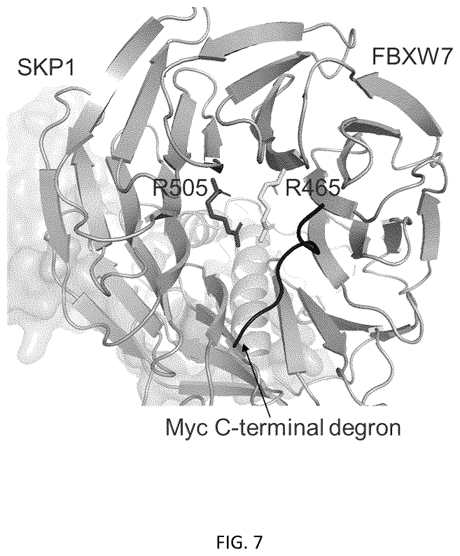

FBXW7-R465C contains two consecutive cysteines (C465 and C466). Since FBXW7 WT does not support 10-SLF-induced FKBP12 degradation, it was hypothesized that C466 itself does not serve as a modified site for inducing target degradation by 10-SLF. Nonetheless, it was next investigated whether C466 might contribute to degradation activity in the context of FBXW7-R465C. To investigate this, a series of mutations in FBXW7, including R465C, R465C/C466A, and C466A (FIG. 3d) were generated, and the ability to support 10-SLF-induced FKBP12 degradation was measured. The results indicated that FBXW7-R465C and FBXW7-R465C/C466A had similar activities in inducing FKBP12 degradation by 10-SLF (FIG. 3d), suggesting a non-essential role for C466 in this process. To gain further insights into the binding model between 10-SLF and FBXW7-R465C, a docking study was conducted using the FBXW7 binding portion of 10-SLF and a crystal structure of FBXW7 (PDB: 7T1Y). The results indicated a favorable fit of 10-SLF on the surface of FBXW7 incorporating the manually introduced R465C mutation. The a-chloroacetamide formed a covalent bond with C465, and its carbonyl group formed a hydrogen bond with R479. Additionally, a hydrogen bond was observed between the oxygen atom of the polyethylene glycol linker and R543. The 4-chlorophenyl group formed a cation-π interaction with R479 and a n-R interaction with W425 (FIG. 3e). Another hotspot mutation, R505C, located near R465C (FIG. 7), was investigated. No FKBP12 degradation by 10-SLF was observed when FBXW7-R505C was expressed (FIG. 3f). As such, the compound 10-SLF appears to rely on FBXW7-R465C, rather than R505C, for target degradation.

### 10-SLF Induces the Formation of a Ternary Complex Between FKBP12 and FBXW7-R465C

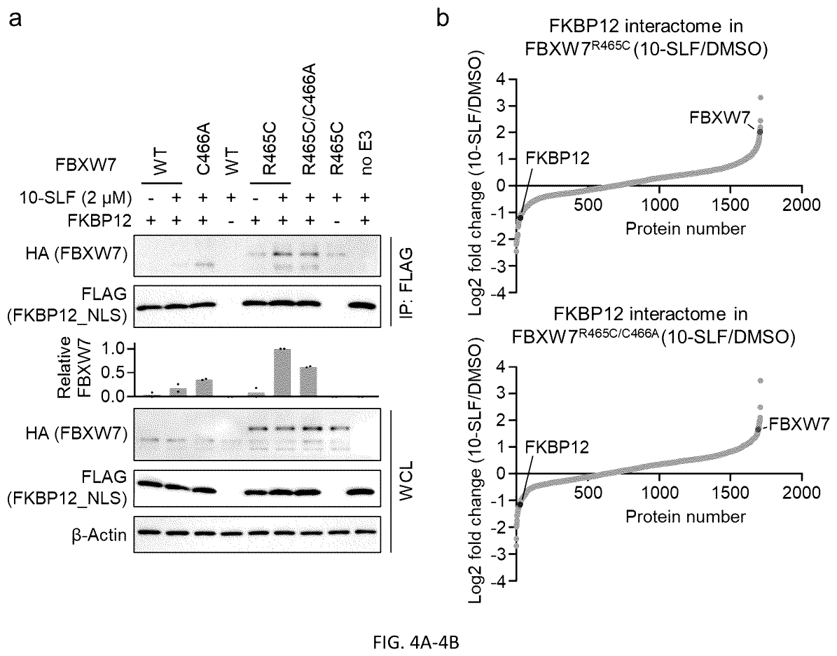

Next, the formation of a ternary complex involving FKBP12, FBXW7-R465C, and 10-SLF, a step that drives target degradation, was evaluated. HEK293T cells expressing FLAG-FKBP12_NLS and HA-FBXW7 variants were treated with 10-SLF and MG132, followed by cell lysis and FKBP12 immunoprecipitation. The results indicated that both FBXW7-R465C and FBXW7-R465C/C466A coimmunoprecipitated with FKBP12 in the presence of 10-SLF (FIG. 4a), supporting the formation of a ternary complex with these mutants and the role of R465C in this process. In contrast, the formation of the ternary complex was significantly hindered when FBXW7 WT or FBXW7-C466A was expressed (FIG. 4a). To further explore the FKBP12 interactome landscape affected by 10-SLF, affinity purification-mass spectrometry (AP-MS) was used to identify proteins interacting with FLAG-FKBP12_NLS from HEK293T cells treated with 10-SLF. The results showed that FBXW7-R465C and FBXW7-R465C/C466A were recruited by 10-SLF (FIG. 4b).

### Utilizing FBXW7-R465C for the Degradation of Additional Protein Targets

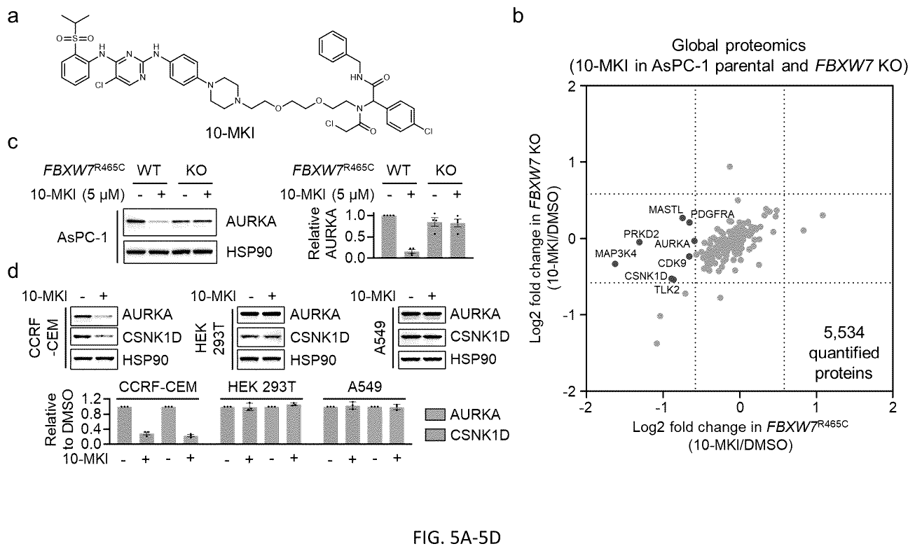

Finally, the potential of harnessing FBXW7-R465C to facilitate the degradation of additional protein targets was assessed. The heterobifunctional compound, 10-MKI, was synthesized by coupling the FBXW7-R465C binding moiety to a multi-kinase binder (FIG. 5a). A global proteomic analysis was conducted in AsPC-1 parental and FBXW7^{R465}C KO cells treated with 10-MKI, identifying a handful of kinases that were more degraded in the parental cells compared to the FBXW7^{R465}C KO cells (FIG. 5b). AURKA was selected as a candidate kinase and degradation by 10-MKI was validated through Western blot analysis in AsPC-1 parental cells, while no degradation was observed in FBXW7^{R465}C KO cells (FIG. 5c). Additionally, three other cell lines were tested: CCRF-CEM (expressing FBXW7^{R465}C) HEK293T (expressing FBXW7 WT), and A549 (expressing FBXW7 WT). Results demonstrated that 10-MKI degraded AURKA and CSNK1D only in the CCRF-CEM cell line expressing FBXW7^{R465}C, while no degradation occurred in the other two cell lines expressing FBXW7 WT (FIG. 5d).

### Example 2

Various PROTACs containing a domain that binds to FBXW7 (e.g. the FBXW7-R465C recruiter) may be generated and tested. For example, various domains that bind to FBXW7 can be conjugated to any suitable target-binding domain and targeted protein degradation of the desired target protein can be evaluated. For example, domains that bind to FBXW7 (e.g. FBXW7-R465C) can be conjugated to a target binding domain for the protein FKBP12, a target binding domain that binds to multiple kinases, such as MKI, a target binding domain that binds to BRD4, a target binding domain that binds to ALK, etc. using a suitable target-binding domain including those described herein. Degradation of the target protein can be assessed in a cell system where FBXW7-R465C and the target protein (e.g. FKBP12, one or more kinases, BRD4, ALK) are co-overexpressed, to identify the top compounds that induce target protein degradation. The top-ranking compounds can then be evaluated for proteome-wide selectivity, such as by using the cysteine-directed activity-based protein profiling (ABPP) platform. From these, the PROTAC that shows the fewest off-target cysteine interactions can be identified. The PROTAC can be assessed using global proteomics in both FBXW7-R465C and wildtype cells to evaluate its degradation potency and proteome-wide selectivity.

The following non-limiting examples of domains that bind to FBXW7 (e.g. FBXW7-R465C) can be used:

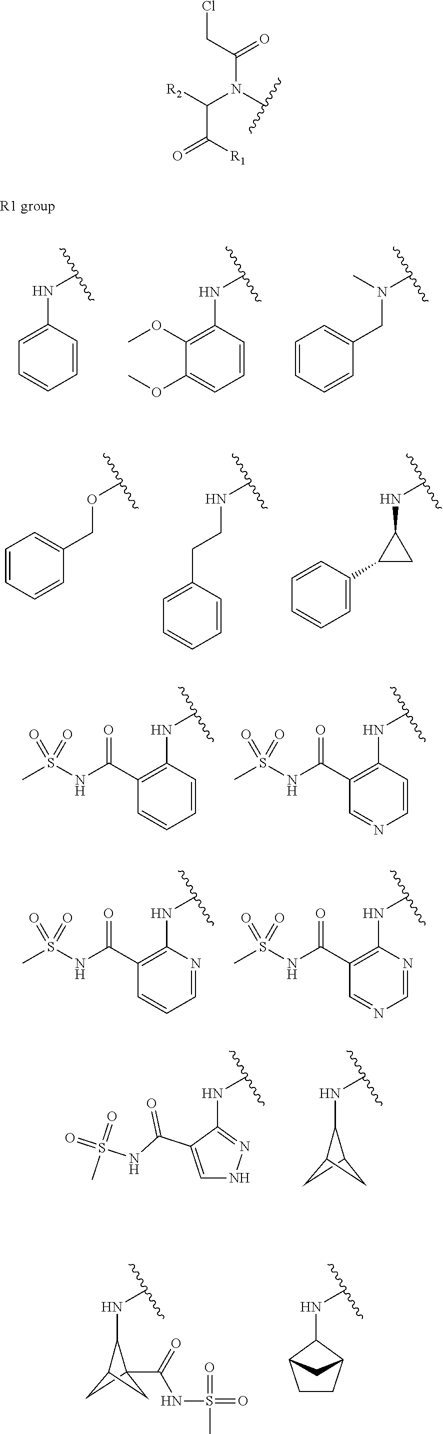

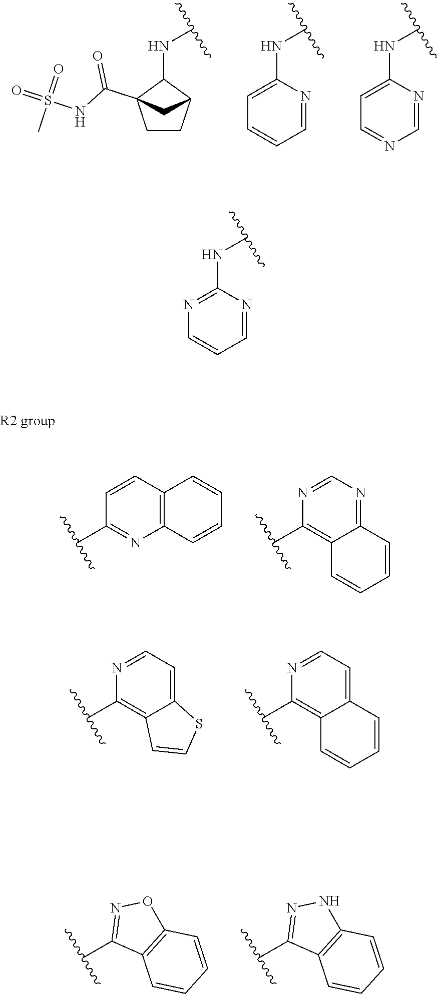

What is claimed is:

1. A proteolysis-targeting chimera (PROTAC) comprising:
a) a domain that binds to the mutant E3 ligase FBXW7-R465C;
b) a target-binding domain, and
c) a linker connecting the domain that binds to FBXW7-R465C to the target-binding domain,
wherein the domain that binds to FBXW7-R465C comprises the structure: 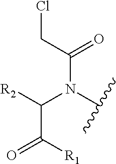
wherein R1 is selected from the following: 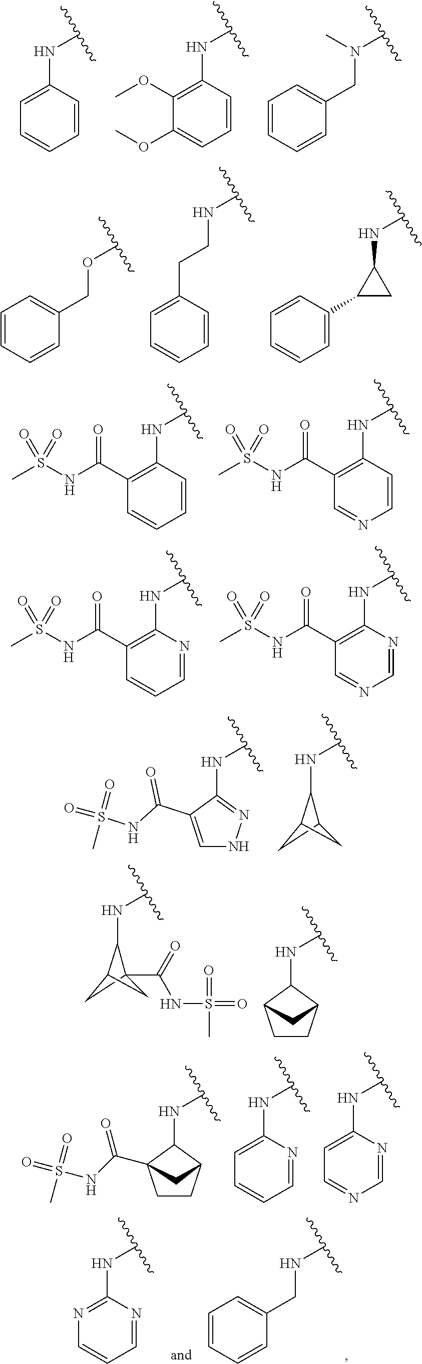
and wherein R2 is selected from the following:

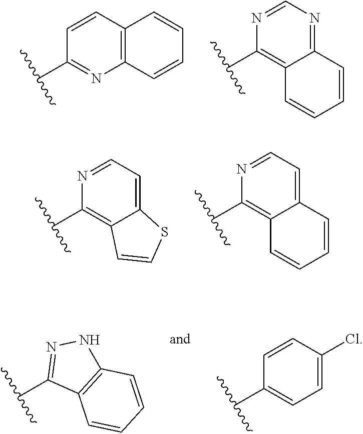

2. The PROTAC of claim 1, wherein the domain that binds to FBXW7-R465C comprises the structure:

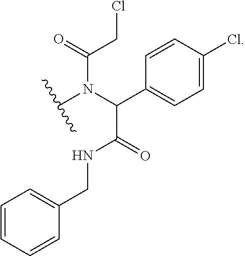 [SMILES: *N(C(=O)CCl)C(C(=O)NCc1ccccc1)c1ccc(Cl)cc1]

3. The PROTAC of claim 1, wherein the linker comprises one or more alkylene oxide units.

4. The PROTAC of claim 1, wherein binding of the PROTAC to FBXW7-R465C and to the target induces FBXW7-R465C-mediated targeted protein degradation of the target.

5. The PROTAC of claim 1, wherein the target is 12-kDa FK506-binding protein (FKBP12).

6. The PROTAC of claim 5, wherein the target-binding domain comprises the structure:

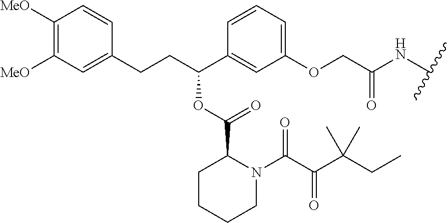 [SMILES: *NC(=O)COc1cccc([C@@H](CCc2ccc(OC)c(OC)c2)OC(=O)[C@@H]2CCCCN2C(=O)C(=O)C(C)(C)CC)c1]

7. The PROTAC of claim 6, wherein the PROTAC is 10-SLF, defined by the structure:

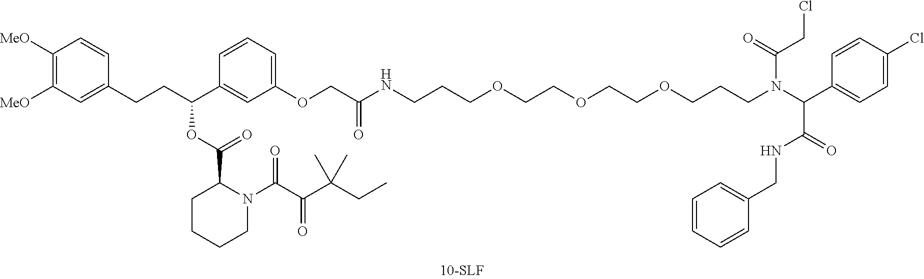 [SMILES: CCC(C)(C)C(=O)C(=O)N1CCCC[C@H]1C(=O)O[C@H](CCc1ccc(OC)c(OC)c1)c1cccc(OCC(=O)NCCCOCCOCCOCCCN(C(=O)CCl)C(C(=O)NCc2ccccc2)c2ccc(Cl)cc2)c1]

8. The PROTAC of claim 1, wherein the target-binding domain binds to one or more kinases.

9. The PROTAC of claim 8, wherein the target-binding domain comprises the structure:

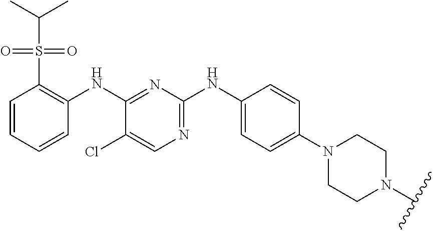 [SMILES: *N1CCN(c2ccc(Nc3ncc(Cl)c(Nc4ccccc4S(=O)(=O)C(C)C)n3)cc2)CC1]

10. The PROTAC of claim 9, wherein the PROTAC is 10-MKI, defined by the structure:

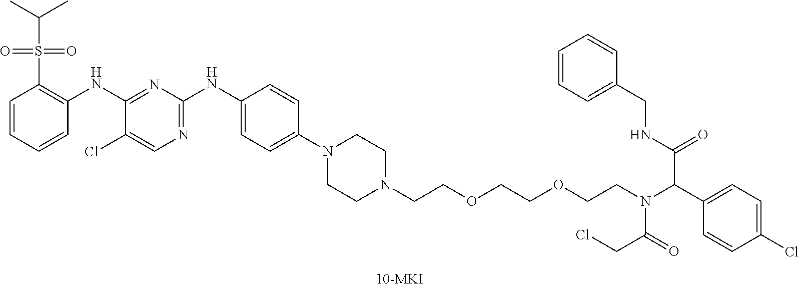 [SMILES: CC(C)S(=O)(=O)c1ccccc1Nc1nc(Nc2ccc(N3CCN(CCOCCOCCN(C(=O)CCl)C(C(=O)NCc4ccccc4)c4ccc(Cl)cc4)CC3)cc2)ncc1Cl]

11. The PROTAC of claim 1, wherein the target is bromodomain-containing protein 4 (BRD4).

12. The PROTAC of claim 11, wherein the target-binding domain comprises the structure:

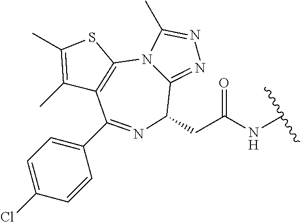 [SMILES: *NC(=O)C[C@@H]1N=C(c2ccc(Cl)cc2)c2c(sc(C)c2C)-n2c(C)nnc21]

13. The PROTAC of claim 1, wherein the target is anaplastic lymphoma kinase (ALK).

14. The PROTAC of claim 13, wherein the target-binding domain comprises the structure:

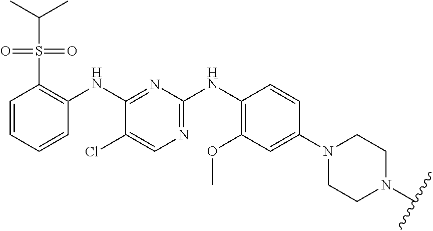 [SMILES: *N1CCN(c2ccc(Nc3ncc(Cl)c(Nc4ccccc4S(=O)(=O)C(C)C)n3)c(OC)c2)CC1]

15. A method for targeted protein degradation in a cell or a subject, the method comprising administering to the cell or subject the PROTAC of claim 1.

16. The method of claim 15, wherein the subject has a neurological disease or cancer.

17. A method for targeted protein degradation of FKBP12 in a subject, comprising administering to the subject the PROTAC of claim 7.

18. The method of claim 17, wherein the subject has a neurological disease.

19. A method for targeted protein degradation of one or more kinases in a subject, comprising administering to the subject the PROTAC of claim 10.

20. The method of claim 19, wherein the subject has cancer.
